(19) United States
(12) Patent Application Publication
(10) Pub. No.: US 2008/0090274 A1
(43) Pub. Date: Apr. 17, 2008

(54) Process For The Synthesis Of (S)-1-(3,5-Bis (Trifluoromethyl)-Phenyl-Ethan-1-Ol

(76) Inventors: Jeffrey C. Moore, Westfield, NJ (US); Matthew D. Truppo, Tinton Falls, NJ (US); Jennifer M. Pollard, Monroe, NJ (US); David J. Pollard, Monroe, NJ (US); Michael G. Sturr, Mountainside, NJ (US)

Correspondence Address: MERCK AND CO., INC, P O BOX 2000, RAHWAY, NJ, 07065-0907, US

(21) Appl. No.: 11/792,612

(22) PCT Filed: Dec. 12, 2005
(86) PCT No.: PCT/US05/45125

§ 371 Date: Jun. 6, 2007

Related U.S. Application Data
(60) Provisional application No. 60/636,735, filed on Dec. 16, 2004.

(51) Int. Cl. C12P 7/22

(52) U.S. Cl. 435/156

(57) ABSTRACT

The present invention is concerned with novel processes for the preparation of (S)-1-(3,5-bis(trifluoromethyl)phenyl)ethan-1-ol (CAS # 30071-93-3). This compound is useful as an intermediate in the synthesis of compounds which possess pharmacological activity.

### BACKGROUND OF THE INVENTION

The present invention relates to processes for the preparation of (S)-1-(3,5-bis(trifluoromethyl)phenyl)ethan-1-ol (CAS # 30071-93-3) which is useful as an intermediate in the preparation of certain therapeutic agents. In particular, the present invention provides a process for the preparation of (S)-1-(3,5-bis(trifluoromethyl)phenyl)ethan-1-ol which is an intermediate in the synthesis of pharmaceutical compounds.

The general processes disclosed in the art for the preparation of (S)-1-(3,5-bis(trifluoromethyl)phenyl)ethan-1-ol result in relatively low and inconsistent yields of the desired product. Some of such processes rely on the use of expensive transition metal catalysts. In contrast to the previously known processes, the present invention provides effective methodology for the preparation of (S)-1-(3,5-bis(trifluoromethyl)-phenyl)ethan-1-ol in relatively high yield and enantiomeric purity.

It will be appreciated that (S)-1-(3,5-bis(trifluoromethyl)phenyl)ethan-1-ol is an important intermediate for a particularly useful class of therapeutic agents. As such, there is a need for the development of a process for the preparation of (S)-1-(3,5-bis(trifluoro-methyl)phenyl)ethan-1-ol which is readily amenable to scale-up, avoids the use of transition metal catalysts, uses cost-effective and readily available reagents, and which is therefore capable of practical application to large scale manufacture.

Accordingly, the subject invention provides a process for the preparation of (S)-1-(3,5-bis(trifluoromethyl)phenyl)ethan-1-ol via a very simple, short and highly efficient synthesis.

### SUMMARY OF THE INVENTION

The novel process of this invention involves the synthesis of (S)-1-(3,5-bis(trifluoromethyl)phenyl)ethan-1-ol. In particular, the present invention is concerned with novel processes for the preparation of a compound of the formula:

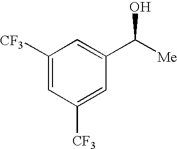

This compound is an intermediate in the synthesis of compounds which possess pharmacological activity. In particular, such compounds are substance P (neurokinin-1) receptor antagonists which are useful e.g., in the treatment of inflammatory diseases, psychiatric disorders, and emesis.

### DETAILED DESCRIPTION OF THE INVENTION

The present invention is directed to processes for the preparation of (S)-1-(3,5-bis(trifluoromethyl)phenyl)ethan-1-ol of the formula:

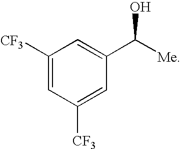

The general process for the preparation of (S)-1-(3,5-bis(trifluoromethyl)-phenyl)ethan-1-ol is as follows:

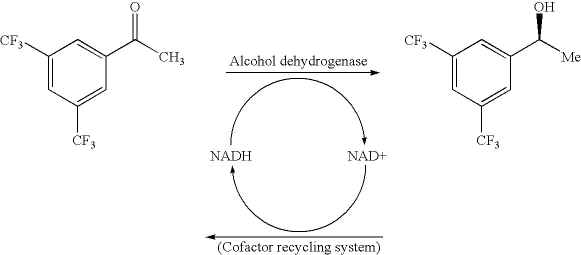

In accordance with this embodiment of the present invention, the treatment of 1-(3,5-bis(trifluoromethyl)-phenyl)ethan-1-one with an alcohol dehydrogenase in the presence of nicotine adenine dinucleotide (NAD) or nicotine adenine dinucleotide phosphate (NADP), and a cofactor recycling system provides (S)-1-(3,5-bis(trifluoromethyl)-phenyl)ethan-1-ol in higher yields, in greater entantiomeric purity and in a more efficient route than the processes disclosed in the art.

An embodiment of the general process for the preparation of (S)-1-(3,5-bis(trifluoromethyl)phenyl)ethan-1-ol is as follows:

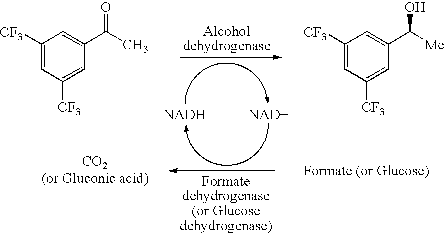

In accordance with this embodiment of the present invention, the treatment of 1-(3,5-bis(trifluoromethyl)-phenyl)ethan-1-one with an alcohol dehydrogenase in the presence of nicotine adenine dinucleotide (NAD), and a cofactor recycling system which comprises: a formate source and a formate dehydrogenase; or a glucose source and a glucose dehydrogenase; provides (S)-1-(3,5-bis(trifluoromethyl)-phenyl)ethan-1-ol in higher yields, in greater entantiomeric purity and in a more efficient route than the processes disclosed in the art.

In an embodiment, the present invention is directed to a process for the preparation of (S)-1-(3,5-bis(trifluoromethyl)phenyl)ethan-1-ol which comprises the treatment of 1-(3,5-bis(trifluoromethyl)-phenyl)ethan-1-one with an alcohol dehydrogenase in the presence of NAD, and a formate source and a formate dehydrogenase to give (S)-1-(3,5-bis(trifluoromethyl)phenyl)ethan-1-ol.

In another embodiment, the present invention is directed to a process for the preparation of (S)-1-(3,5-bis(trifluoromethyl)phenyl)ethan-1-ol which comprises the treatment of 1-(3,5-bis(trifluoromethyl)-phenyl)ethan-1-one with an alcohol dehydrogenase in the presence of NAD, and a glucose source and a glucose dehydrogenase to give (S)-1-(3,5-bis(trifluoromethyl)phenyl)ethan-1-ol.

A specific embodiment of the present invention concerns a process for the preparation of (S)-1-(3,5-bis(trifluoromethyl)phenyl)ethan-1-ol of the formula: 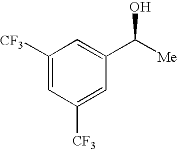
which comprises:

treating 1-(3,5-bis(trifluoromethyl)phenyl)ethan-1-one of the formula: 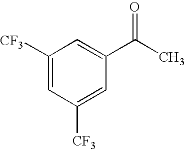
with an alcohol dehydrogenase in the presence of nicotine adenine dinucleotide and a cofactor recycling system; to give (S)-1-(3,5-bis(trifluoromethyl)phenyl)ethan-1-ol of the formula:

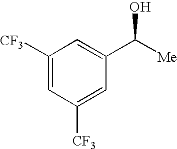

Another embodiment of the present invention concerns a process for the preparation of (R)-1-(3,5-bis(trifluoromethyl)phenyl)ethan-1-ol of the formula: 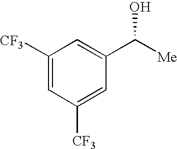
which comprises:

treating 1-(3,5-bis(trifluoromethyl)phenyl)ethan-1-one of the formula: 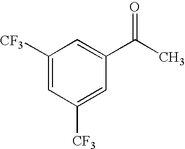
with an alcohol dehydrogenase in the presence of nicotine adenine dinucleotide and a cofactor recycling system; to give (R)-1-(3,5-bis(trifluoromethyl)phenyl)ethan-1-ol of the formula:

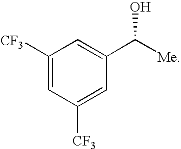

In the present invention, the cofactor recycling system includes those which comprise: a formate source and a formate dehydrogenase; or a glucose source and a glucose dehydrogenase.

In the present invention, the alcohol dehydrogenase includes those selected from: alcohol dehydrogenase from Rhodococcus erythropolis; alcohol dehydrogenase from Candida parapsilosis; and alcohol dehydrogenase from Candida boidinii. In the present invention, the alcohol dehydrogenase may be present at a concentration of about 3-7 KU/L (Kilo Units/Liter). In the present invention, the alcohol dehydrogenase may be present at a concentration of about 3 KU/L. Kilo Units (KU) are standard units for measuring enzyme activity. These units of standard activity of enzymes are well understood by persons skilled in the art.

In the present invention, the formate source includes those selected from sodium formate and formic acid. In the present invention, the formate source may be present at a concentration of about 500 mM.

In the present invention, the formate dehydrogenase includes those selected from formate dehydrogenase. In the present invention, the formate dehydrogenase may be present at a concentration of about 2.9-3.8 KU/L (Kilo Units/Liter) (or 0.7-1 g/L). In the present invention, the formate dehydrogenase may be present at a concentration of about 2.9 KU/L (or 0.7 g/L).

In the present invention, the nicotine adenine dinucleotide (NAD) may be present at a concentration of about 0.7-1 g/L. In the present invention, the nicotine adenine dinucleotide may be present at a concentration of about 1 g/L.

In the present invention, the glucose source includes those selected from glucose. In the present invention, the glucose source may be present at a concentration of about 450-600 mM.

In the present invention, the glucose dehydrogenase includes those selected from glucose dehydrogenase 103 (Biocatalytics). In the present invention, the glucose dehydrogenase may be present at a concentration of about 2.1-4.2 KU/L (Kilo Units/Liter) (or 0.035-0.7 g/L).

In the present invention, the reaction mixture may comprise an aqueous buffer, such as a phosphate buffer. In the present invention, the reaction mixture may further comprise an organic solvent, such as heptane, hexane or pentane. In an embodiment of the present invention, the reaction mixture may further comprise an organic solvent which is heptane. In an embodiment of the present invention, the organic solvent may be present at a concentration of 0-5% v/v.

In an embodiment of the present invention, the pH of the reaction mixture is maintained between pH 6-8. In an embodiment of the present invention, the pH of the reaction mixture is maintained between pH 6.5-7.5. In an embodiment of the present invention, the pH of the reaction mixture is maintained between pH 6.8-7.3, such as by the addition of an acid or base.

In an embodiment of the present invention, the temperature of the reaction mixture is maintained at about 26-33 deg C. In a further embodiment of the present invention, the temperature of the reaction mixture is maintained at about 30 deg C.

For convenience, the alcohol dehydrogenase, NAD, and a formate source and a formate dehydrogenase may be contacted together in situ, prior to reaction with (S)-1-(3,5-bis(trifluoromethyl)phenyl)ethan-1-ol. Likewise for convenience, the alcohol dehydrogenase, NAD, and a glucose source and a glucose dehydrogenase, may be contacted together in situ, prior to reaction with (S)-1-(3,5-bis(trifluoromethyl)phenyl)ethan-1-ol.

The (S)-1-(3,5-bis(trifluoromethyl)phenyl)ethan-1-ol obtained in accordance with the present invention may be used as starting material in further reactions directly or following purification.

In a further embodiment, the present invention is directed to a process for purification or for enhancing the enantiomeric purity of (S)-1-(3,5-bis(trifluoromethyl)-phenyl)ethan-1-ol which comprises:

extracting the reaction mixture with a solvent which comprises heptane; concentrating the solvent; and crystallizing (S)-1-(3,5-bis(trifluoromethyl)-phenyl)ethan-1-ol.

In an aspect of this further embodiment, extracting the reaction mixture with a solvent which comprises heptane is conducted at a temperature of about 50-55 deg C.

In an alternate aspect of this further embodiment, the reaction mixture is extracted with a solvent which comprises heptane, and further comprises methanol, ethanol or ethyl acetate.

Within this alternate aspect, the reaction mixture is extracted with a solvent which comprises heptane and methanol. For example, the methanol may be present at a concentration of about 10% (v/v).

Within this alternate aspect, the reaction mixture is extracted with a solvent which comprises heptane and ethanol. For example, the ethanol may be present at a concentration of about 5-10% (v/v).

Within this alternate aspect, the reaction mixture is extracted with a solvent which comprises heptane and ethyl acetate. For example, the ethyl acetate may be present at a concentration of about 5-10% (v/v).

In an aspect of this further embodiment, concentrating the solvent is conducted by vacuum distillation at a temperature of about 40-45 deg C.

In an aspect of this further embodiment, crystallizing the (S)-1-(3,5-bis(tri-fluoromethyl)phenyl)ethan-1-ol is conducted at a temperature of between about 45 deg C. and about −10 deg C. Within this alternate aspect, seed crystals of (S)-1-(3,5-bis(tri-fluoromethyl)phenyl)ethan-1-ol are added to the concentrated solvent. Further within this alternate aspect, seed crystals of (S)-1-(3,5-bis(tri-fluoromethyl)phenyl)ethan-1-ol are present at a concentration of 0.5-1% gram seed/gram of substrate.

It will be appreciated by those skilled in the art that this alternate embodiment may be repeated in an iterative manner to further enhance the enantiomeric purity of (S)-1-(3,5-bis(trifluoromethyl)-phenyl)ethan-1-ol with each subsequent cycle.

Another aspect of this invention is directed to (S)-1-(3,5-bis(trifluoro-methyl)phenyl)ethan-1-ol which is present in an enantiomeric purity (enantiomeric excess) of greater than 90%, greater than 95%, greater than 98%, greater than 99%, greater than 99.5% (enantiomeric excess) or greater than 99.9% (enantiomeric excess).

The starting materials and reagents for the subject processes are either commercially available or are known in the literature or may be prepared following literature methods described for analogous compounds (see for example, U.S. Pat. Nos. 6,255,545, 6,350,915 and 6,814,895). 3,5-Bis(trifluoromethyl)bromobenzene (CAS 328-70-1) and 1-(3,5-bis(trifluoromethyl)phenyl)ethan-1-one (CAS 30071-93-3) are commercially available. The skills required in carrying out the reaction and purification of the resulting reaction products are known to those in the art. Purification procedures include crystallization, distillation, normal phase or reverse phase chromatography.

The following examples are provided for the purpose of further illustration only and are not intended to be limitations on the disclosed invention.

### EXAMPLE 1

### (S)-1-(3,5-Bis(trifluoromethyl)phenyl)ethan-1-ol

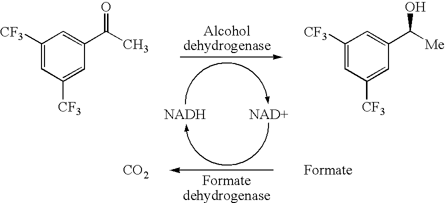

The enzyme reaction used 50 mM phosphate buffer pH 7.0. Sodium formate (500 mM) and NAD (1 g/L) were dissolved in the buffer followed by the addition of the enzymes (RE alcohol dehydrogenase (3 KU/L) and formate dehydrogenase (0.7 g/L or 2.88 KU/L)). 1-(3,5-Bis(trifluoromethyl)phenyl)ethan-1-one (CAS 30071-93-3) was added to the reaction as a single solution (100 g/L). pH was controlled at pH 7.0 using 2N sulphuric acid. Reaction was run for 28 to 40 hours at 30 deg C. Conversion >95% was usually achieved by 40 hours with enantiomeric excess >99%.

The product was isolated by two ½ volume extractions in heptane at 50 deg C., followed by ¼ volume water wash and vacuum concentration by distillation (2-3 fold volume concentration at 40 deg C.). For crystallization the solution was cooled from 45 deg C. to 35 deg C. (200 g/L alcohol concentration in heptane). Seeding with (S)-1-(3,5-bis(trifluoromethyl)phenyl)ethan-1-ol at 1% g/gram of substrate was completed at 35 deg C., followed by 1 hour of aging and cool down to −10 deg C. The crystallization procedure rejects impurities such as residual ketone. Final material purity >99% was produced with Enantiomeric excess >99%.

### EXAMPLE 2

### (S)-1-(3,5-Bis(trifluoromethyl)phenyl)ethan-1-ol

### Alternate Process

The enzyme reaction used 50 mM phosphate buffer pH 7.0. Sodium formate (500 mM) and NAD (0.7-1 g/L) were dissolved in the buffer followed by the addition of the enzymes (RE alcohol dehydrogenase (3-7 KU/L), formate dehydrogenase (0.7-1 g/L or 2.9-3.74 KU/L)) and heptane (0-5% v/v). 1-(3,5-Bis(trifluoromethyl)phenyl)ethan-1-one was added to the reaction as a single solution (10-110 g/L). pH was controlled between pH 6.8-7.3 using 2N sulphuric acid. Reaction was run for 28 to 40 hours at 26-33 deg C. Conversion >95% was achieved by 40 hours with enantiomeric excess >99%.

The product was isolated by two ½-1 volume extractions in heptane at 50-55 deg C., followed by ¼-1 water wash and concentration by vacuum distillation (40-55 deg C.) with a 2-3 fold concentration. For crystallization the solution was cooled from 45 deg C. to 35 deg C. (80 g/L-200 g/L alcohol concentration in heptane). Seeding with (S)-1-(3,5-bis(trifluoro-methyl)phenyl)ethan-1-ol at 0.5-1% g/gram of substrate is completed at 35 deg C., followed by 1 hour of aging and cool down to −10 deg C. The crystallization procedure rejects impurities such as residual ketone (upto 40% ketone rejection). The product was dried at room temperature and full vacuum. Final material purity >99% was produced with EE >99%.

In an alternate embodiment, the process may performed by replacing the alcohol dehydrogenase (ADH) from Rhodococcus erythropolis with the ADH from Candida parapsilosis or ADH from Candida boidinii.

### EXAMPLE 3

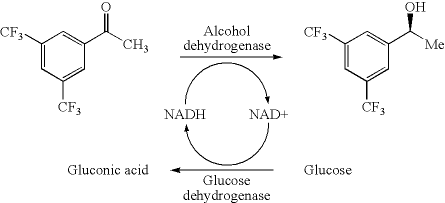

### (S)-1-(3,5-Bis(trifluoromethyl)phenyl)ethan-1-ol

### Alternate Process

The enzyme reaction uses 50 mM phosphate buffer pH 7.0. Glucose (450-600 mM) and NAD (0.7-1 g/L) were dissolved in the buffer followed by the addition of the enzymes (RE alcohol dehydrogenase (3-7 KU/L), glucose dehydrogenase 103 (Biocatalytics) (0.035-0.7 g/L or 2.1-4.2 KU/L)) and heptane (0-5% v/v). 1-(3,5-Bis(trifluoromethyl)-phenyl)ethan-1-one was added to the reaction as a single solution (10-110 g/L). pH was controlled between pH 6.8-7.3 using 2N sulphuric acid. Reaction was run for 20-30 hours at 26-33 deg C. Conversion >95% was achieved by 20 hours with enantiomeric excess >99%.

The product was isolated by three ½ volume extractions in heptane with ethanol 15% or methanol 10% or 5-10% ethyl acetate at 25 deg C., followed by ¼-1 water wash and vacuum concentration by distillation (40-55 deg C.) with a 2-3 fold concentration. For crystallization the solution was cooled from 45 deg C. to 35 deg C. (80 g/L-200 g/L alcohol concentration in heptane). Seeding with (S)-1-(3,5-bis(trifluoromethyl)phenyl)-ethan-1-ol at 0.5-1% g/gram of substrate was completed at 35 deg C., followed by 1 hour of aging and cool down to −10 deg C. The crystallization procedure rejects impurities such as residual ketone (upto 20% ketone rejection). The product was dried at room temperature and full vacuum: Final material purity >99% was produced with EE >99%.

### EXAMPLE 4

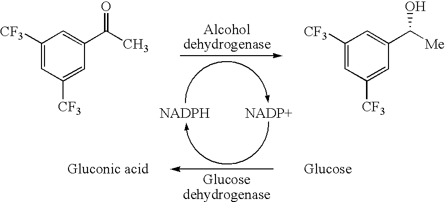

### (R)-1-(3,5-Bis(trifluoromethyl)phenyl)ethan-1-ol

The route to (R)-1-(3,5-bis(trifluoromethyl)phenyl)ethan-1-ol is shown above. Recycling of the required NADPH cofactor is completed using glucose dehydrogenase with glucose. The enzyme reaction uses 200 mM phosphate buffer (pH 7) with 500 mM glucose and NADP at 1-2 g/L. The oxidoreductase is KRED 101 from Biocatalytics Inc at 10-20 kU/L. Glucose dehydrogenase is used to recycle the cofactor. Ketone is added to the reaction as a solution and pH controlled at pH 7 by 2N sulphuric acid. Reaction time is around 30-40 hours at 30 deg C. with enantiomeric excess of >99%. The (R)-1-(3,5-bis(trifluoromethyl)phenyl)ethan-1-ol is isolated by any of the procedures described for the (S) alcohol routes above.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, reaction conditions other than the particular conditions as set forth herein above may be applicable as a consequence of variations in the reagents or methodology to prepare the compounds from the processes of the invention indicated above. Likewise, the specific reactivity of starting materials may vary according to and depending upon the particular substituents present or the conditions of manufacture, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A process for the preparation of a compound of the formula: 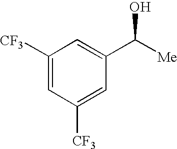
which comprises:
treating 1-(3,5-bis(trifluoromethyl)phenyl)ethan-1-one of the formula: 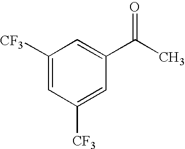
with an alcohol dehydrogenase in the presence of nicotine adenine dinucleotide and a cofactor recycling system; to give the compound of the formula:

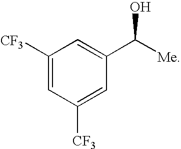

2. The process of claim 1 wherein the alcohol dehydrogenase is selected from:
alcohol dehydrogenase from Rhodococcus erythropolis; alcohol dehydrogenase from Candida parapsilosis; and alcohol dehydrogenase from Candida boidinii.

3. The process of claim 2 wherein the alcohol dehydrogenase is alcohol dehydrogenase from Rhodococcus erythropolis.

4. The process of claim 1 wherein the alcohol dehydrogenase is present at a concentration of about 3-7 KU/L.

5. The process of claim 1 wherein the cofactor recycling system comprises: a formate source and a formate dehydrogenase; or a glucose source and a glucose dehydrogenase.

6. The process of claim 5 wherein the cofactor recycling system further comprises nicotine adenine dinucleotide.

7. The process of claim 6 wherein the nicotine adenine dinucleotide is present at a concentration of about 0.7-1 g/L.

8. The process of claim 5 wherein the cofactor recycling system comprises: a formate source and a formate dehydrogenase.

9. The process of claim 8 wherein the formate source is selected from sodium formate and formic acid.

10. The process of claim 9 wherein the formate source is sodium formate.

11. The process of claim 8 wherein the formate source is present at a concentration of about 500 mM.

12. The process of claim 8 wherein the formate dehydrogenase is present at a concentration of about 2.9-3.8 KU/L.

13. The process of claim 12 wherein the formate dehydrogenase is present at a concentration of about 2.9 KU/L.

14. The process of claim 5 wherein the cofactor recycling system is selected from: a glucose source and a glucose dehydrogenase.

15. The process of claim 14 wherein the glucose source is glucose.

16. The process of claim 14 wherein the glucose source is present at a concentration of about 450-600 mM.

17. The process of claim 14 wherein the glucose dehydrogenase is glucose dehydrogenase.

18. The process of claim 14 wherein the glucose dehydrogenase is present at a concentration of about 2.1-4.2 KU/L.

19. The process of claim 1 wherein the reaction mixture comprises a phosphate buffer.

20. The process of claim 1 wherein the reaction mixture further comprises an organic solvent which is heptane.

21. The process of claim 1 which further comprises:
extracting the reaction mixture with a solvent which comprises heptane; concentrating the solvent; and crystallizing the compound of the formula:

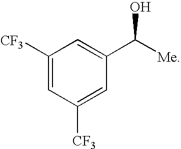

22. The process of claim 21 which comprises extracting the reaction mixture with a solvent which comprises heptane at a temperature of about 50-55 deg C.

23. The process of claim 21 which comprises extracting the reaction mixture with a solvent which comprises heptane, and further comprises methanol, ethanol or ethyl acetate.

24. The process of claim 21 wherein the step of concentrating the solvent is conducted by vacuum distillation at a temperature of about 40-45 deg C.

25. The process of claim 21 wherein the step of crystallizing the compound is conducted at a temperature of between about 45 deg C. and about −10 deg C.
